(19) United States
(12) Patent Application Publication
(10) Pub. No.: US 2023/0390168 A1
(43) Pub. Date: Dec. 7, 2023

(54) COMPOSITION FOR CONDITIONING KERATIN MATERIALS

(71) Applicant: L'OREAL, Paris (FR)

(72) Inventors: Yuanji GUO, Shanghai (CN); Shichen ZHANG, Shanghai (CN)

(73) Assignee: L'OREAL, Paris (FR)

(21) Appl. No.: 18/250,875

(22) PCT Filed: Oct. 30, 2020
(86) PCT No.: PCT/CN2020/125377

§ 371 Date: Apr. 27, 2023

(51) Int. Cl. A61K 8/25; A61K 8/02; A61K 8/34; A61K 8/73; A61Q 5/12; A61Q 19/00

(52) U.S. Cl. CPC A61K 8/25; A61K 8/025; A61K 8/34; A61K 8/731; A61Q 5/12; A61Q 19/00; A61K 2800/49; A61K 2800/5426; A61K 2800/34; A61K 2800/48

(57) ABSTRACT

It relates to a composition for conditioning keratin materials, comprising (A) a spheric particles of lipophilically modified silica; (B) a medium/solvent, selected from water and alcohol, and (C) at least one conditioning active agent.

### TECHNICAL FIELD

The present invention relates to a composition for conditioning keratin materials, in particular the skin, scalp or hair along with scalp. The invention also relates to a process for conditioning keratin materials, in particular the skin, scalp or hair along with scalp, using the composition of the present invention.

### BACKGROUND

Keratin materials are present widely on surface of human bodies, e.g., skin, scalp and hair. Many people have for a long time sought to fresh the body surface, so as to feel comfortable in daily life and work. Amongst others, the feeling of sticky/greasy is particularly concerned by many people. Accordingly, various products are developed to deal with the sticky or greasy feeling of person.

In the keratin materials on body surface, the scalp is particularly readily to suffer from grease. Various formulations have been developed for the sticky or greasy scalp, including those provided in a separate form, e.g., lotion, spray and the like, and those incorporated into conventional products for hair and/or for scalp, e.g., shampoo, leave-on and/or rinse-off conditioner.

Various conditioning compositions have been developed mainly for hair, which are generally used to improve or return to the hair its natural luster, shine, smoothness, and softness. When used with shampooing, the commonly accepted method for conditioning the hair has been to shampoo the hair, followed by rinsing the hair, and then applying a conditioner composition, optionally followed by a second rinse.

There is thus still needed in the art to develop new compositions for caring mainly for scalp, especially the non-greasy feeling.

### SUMMARY OF THE INVENTION

Silica is known to be used in cosmetics, e.g., as a conventional filler, or as a substrate to absorb dyes for lake. Derivatives of silica and/or modified silica are also used. However, it is unknown in the art about any roles of (modified) silica solve of the greasy status of keratin materials. By deep study, the inventors have surprisingly discovered that by using specifically modified silica, especially modification in view of both structure and chemical bonds, in a specific form, the sebum migration, and accordingly the greasy feeling, on keratin materials, in particular on scalp, can be effectively inhibited.

Thus, one subject of the present invention is thus a leave-on composition for conditioning keratin materials, comprising:
spheric particles of lipophilically modified silica homogeneously dispersed in the medium/solvent; and
at least one medium/solvent.

The composition according to the present invention is particularly useful for conditioning keratin materials, such as, in particular for scalp care. Accordingly, the composition of the present invention can be in various form, e.g., lotion, cream, spray and the like to be applied to scalp directly, to skin or to hair along with scalp.

### EMBODIMENTS OF THE INVENTION

Throughout the description, including the claims, the term “comprising a” should be understood as being synonymous with “comprising at least one”, unless otherwise mentioned. Moreover, the expression “at least one” used in the present description is equivalent to the expression “one or more”.

Throughout the description, including the claims, an embodiment defined with “comprising” or the like should be understood to encompass a preferable embodiment defined with “consisting substantially of” and a preferable embodiment defined with “consisting of”.

Other than in the operating examples, or where otherwise indicated, all numbers expressing quantities of components and/or reaction conditions are to be understood as being modified in all instances by the term “about”, with conventionally known meaning in the art, e.g., within 10% of the indicated number (e.g. “about 10%” means 9%-11% and “about 2%” means 1.8%-2.2%).

Throughout the description, including the claims, the “keratin material” according to the present invention is the skin, hair, scalp, eyelashes, eyebrows, bodily hair, nails, lips or mucous membranes. Preferably, the keratin material according to the present invention is a position where an sticky or greasy feeling is easily felt, e.g., scalp, or skin, and/or a position where the sticky or greasy feeling has an impact on, e.g., hair.

Throughout the description, including the claims, the “care for”, “caring for” and the like according to the present invention should be understood to encompass any means to take care, maintain or improve the status of keratin materials, including not only the conventional means of caring for keratin materials, e.g., skin or scalp, but also any means known to be useful for conditioning and/or cleansing keratin materials, e.g., hair or scalp.

In the application, unless specifically mentioned otherwise, contents, parts and percentages are expressed on a weight basis.

The present invention is directed to a composition for caring for keratin materials, comprising:
(A) spheric particles of lipophilically modified silica homogeneously dispersed in the medium/solvent;
(B) at least one medium/solvent selected from water and alcohols; and
(C) at least one conditioning active agent.

The present invention thus provides use of the spheric particles of lipophilically modified silica for soothing the feeling of greasy of keratin materials on body surface, especially scalp.

Other characteristics and advantages of the invention will emerge more clearly on reading the description and the examples that follow.

Spheric Particles of Lipophilically Modified Silica (A)

The composition of the present invention comprises spheric particles of lipophilically modified silica (A).

According to the present invention, a “spheric particle” means a particle in the shape of sphere or at least approximate sphere, and/or a particle having an aspect ratio of about 0.9 to 1.1, e.g., about 0.95 to 1.05. For example, those skilled in the art can determine whether a particle is in the shape of sphere according to the conventional knowledge. In particular, regarding a given silica, it is within the ordinary skill of persons in the art to distinguish the shape of sphere from other shapes of plate, rod, powder, strip, irregular shapes or the like, and from amorphous similar.

According to the present invention, a “homogeneously dispersed” state is desired for the silica. For example, the formation of dispersion, emulsion, solution or the like, having known definition and/or criteria in the art, can represent the achievement of a “homogeneously dispersed” state. Nonetheless, it is to be understood that once a state can be determined by those skilled in the art as being distinguished significantly from a “non-homogeneously dispersed” state, the state can be deemed as substantially satisfying the “homogeneously dispersed” state.

According to the present invention, a “lipophilically modified” silica means one having subjected to lipophilic modification. According to the present invention, lipophilic modification means any organo-modification capable of bringing hydrophobic or lipophilic treatment to the silica. For example, a silica can be treated with a siloxane compound, e.g., hydroxydimethylsiloxane (or other silanes, such as silazane, etc.), through which the silanol group on the surface of the silica and the silanol group of the siloxane compound are condensed. Amongst others, useful examples of “lipophilically modified” silica can be made to silica silylate and silica dimethyl silylate, e.g., silica silylate, such as the one available from company TOKUYAMA CORPORATION under the name of AIRLICA® TL-10.

According to the present invention, when being incorporated into the composition, the spheric particles of lipophilically modified silica can show an oil-absorbing capacity of 500 ml/100 g or more, 600 ml/100 g or more, or even 700 ml/100 g or more.

The spheric particles of lipophilically modified silica (A) may be present in the composition according to the present invention in an amount ranging from 0.01% to 5% by weight, preferably ranging from 0.1% to 3% by weight, or preferably ranging from 0.3% to 1% by weight, relative to the total weight of the composition.

Medium/Solvent (B)

The composition according to the invention can advantageously comprise at least one medium/solvent for use as component (B), including water and/or organic medium/solvent. For the purpose of the present invention, the component (B) serves as medium for component (A), the spheric particles of lipophilically modified silica, and if present, other insoluble components to be dispersed as homogeneously as possible, and serves as solvent for the soluble components if present.

Water

The composition according to the invention may advantageously comprise water in various amounts. For low viscosity applications of the composition, e.g., in form of leave-on lotion, a relatively high amount of water may be used. For example, water is used in a content of greater than or equal to 40% by weight relative to the total weight of composition. The water content in the low viscosity composition according to the invention preferably ranges from 40% to 99% by weight, more preferably from 50% to 90% by weight, or from 60% to 80% by weight, relative to the total weight of the composition.

For high viscosity applications of the composition, e.g., in form of leave-on cream, a relatively lower amount of water may be used. The high viscosity composition according to the invention advantageously comprises water in a content of less than or equal to 40% by weight relative to the total weight of composition (I). The water content in the low viscosity composition according to the invention preferably ranges from 10% to 40% by weight, more preferably from 15% to 35% by weight, or from 20% to 30% by weight, relative to the total weight of the composition.

Organic Medium/Solvent

The composition according to the invention may also comprise one or more organic medium/solvents, preferably water-soluble organic medium/solvents (solubility of greater than or equal to 5% in water at 25° C. and at atmospheric pressure).

Examples of the organic medium/solvents that may be mentioned include linear or branched, and preferably saturated, monoalcohols or diols, comprising 2 to 10 carbon atoms, such as ethanol, (iso)propanol, butanol, hexylene glycol (2-methyl-2,4-pentanediol), neopentyl glycol and 3-methyl-1,5-pentanediol, butylene glycol, dipropylene glycol and propylene glycol; aromatic alcohols such as phenylethyl alcohol; polyols containing more than two hydroxyl functions, in particular C3-C6 polyols, such as glycerol; polyol ethers, for instance ethylene glycol monomethyl, monoethyl and monobutyl ether, propylene glycol or ethers thereof, for instance propylene glycol monomethyl ether; and also diethylene glycol alkyl ethers, especially C₁-C₄ alkyl ethers, for instance diethylene glycol monoethyl ether or monobutyl ether, alone or as a mixture.

The organic medium/solvents, when they are present, generally represent between 0.1% and 40% by weight relative to the total weight of the composition according to the invention, and preferably between 1% and 30% by weight, or between 5% and 20% by weight.

Conditioning Active Agent (C)

The composition according to the invention may also comprise at least one conditioning active agent (C), which benefits the conditioning effect, especially for a leave-on conditioner product useful for keratin materials.

The conditioning active agent (C) of the present invention may refer to ingredients that enhance the appearance and feel of scalp/hair, by increasing hair body, suppleness, or sheen, or by improving the texture of hair that has been damaged physically or by chemical treatment. The conventional scalp/hair conditioning active agents are well adapted to the present invention. Such conditioning active agents include non-silicone cationic surfactants, silicones, fatty esters, and other ingredients which are known in the art as having desirable hair conditioning properties.

Among others, cationic surfactants are widely used as a conditioning agent. The ability of this kind of cationic surfactants to provide conditioning effect to hair is attributable to the hydrophobic nature of the alkyl chain as well as to the cationic charge of the polar head group. Examples of cationic surfactants known are quaternary ammonium compounds containing in the molecule at least one long alkyl chain. The term “non-silicone cationic surfactant” means a surfactant having no silicone atoms in its structure and positively charged when it is contained in the composition of the invention. This surfactant may carry one or more permanent positive charges or contain one or more cationizable functions in the composition of the invention.

When the conditioning agent is a fatty ester, it may either be an ester of a C8-C30 fatty acid and C1-C30 monoalcohols or polyols including esters of C8-C30 fatty acids and C8-C30 fatty alcohols, or an ester of a C1-C7 dibasic acid and a C8-C30 fatty alcohol.

The conditioning active agent (C) of the present invention may also be those providing cooling and/or freshness feeling, such as menthol and derivatives thereof, e.g., ethyl menthane carboxamide, menthyl lactate, methoxypropanediol.

The conditioning active agent (C) may be present in the composition according to the present invention in an amount ranging from 0.001% to 1% by weight, preferably ranging from 0.01% to 0.5% by weight, or preferably ranging from 0.02% to 0.2% by weight, relative to the total weight of the composition.

Thickening Agent (D)

As a leave-on form, it is preferable that the leave-on product can be distributed uniformly on scalp, or hair along with scalp. Accordingly, at least one thickening agent (D) can be preferably incorporated into the composition according to the present invention, so as to adjust the viscosity of the product obtained, and to facilitate lamellar distribution or the formation of a uniform film of the composition on scalp, or hair along with scalp.

Thickening agents (D) include those which increase the viscosity of the composition without significantly altering the effectiveness of one or more of the active ingredients within the composition. Thickening agents can also increase the compositional stability of the composition.

Non-limiting examples of thickening agents (D) include carboxylic acid polymers, crosslinked polyacrylate polymers, polyacrylamide polymers, polysaccharides, and gums.

Examples of the carboxylic acid polymer include a crosslinking compound containing one or more monomers derived from acrylic acid, substituted acrylic acid, and salts and esters of these acrylic acid and substituted acrylic acid, wherein the crosslinking agent contains two or more carbons. The carbon double bond is derived from a polyol. Examples of commercially available carboxylic acid polymers include carbomer, which is a homopolymer of acrylic acid crosslinked with sucrose or an allyl ether of pentaerythritol (e.g., CARBOPOL® ULTREZ 10 POLYMER from LUBRIZOL).

Non-limiting examples of polyacrylamide polymers, including nonionic polyacrylamide polymers, including substituted branched or unbranched polymers, include polyacrylamides, isoparaffins, and laureth-7, acrylamide. And substituted multi-block copolymers of acrylamide with acrylic acid and substituted acrylic acid.

Non-limiting examples of polysaccharides include cellulose, carboxymethyl hydroxyethyl cellulose, cellulose acetate propionate, hydroxyethyl cellulose, hydroxyethyl ethyl cellulose, hydroxypropyl cellulose, hydroxypropyl Methylcellulose, methylhydroxyethylcellulose, microcrystalline cellulose, sodium cellulose sulfate, and mixtures thereof. Another example is an alkyl substituted cellulose wherein the hydroxyl group of the cellulosic polymer is hydroxyalkylated (preferably hydroxyethylated or hydroxypropylated) to form a hydroxyalkylated cellulose, which is subsequently C₁₀-C₃₀ straight or branched alkyl group is further modified by an ether linkage. These polymers are typically ethers of C₁₀-C₃₀ straight or branched alcohols with hydroxyalkyl celluloses. Other useful polysaccharides include linear scleroglucans comprising (1-3) linked glucose units, wherein each of the three units has (1-6) linked glucose.

Non-limiting examples of gums include gum arabic, agar, algin, alginic acid, ammonium alginate, amylopectin, calcium alginate, calcium carrageenan, carnitine, carrageenan, dextrin, gelatin, guar gum, guar hydroxypropyltrimonium chloride, hectorite, hyaluronic acid, hydrated silica, hydroxypropyl chitosan, hydroxypropyl guar, karaya gum, seaweed, locust bean gum, natto gum, potassium alginate, carrageenan potassium, propylene glycol alginate, sclerotium gum, sodium carboxymethyl dextran, carrageenan sodium, tragacanth, xanthan gum and mixtures thereof.

Other examples of polymeric thickening agents that may be mentioned include vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol.

The thickening agent may be present in the composition according to the present invention in an amount ranging from 0.01% to 5% by weight, preferably ranging from 0.05% to 3% by weight, or preferably ranging from 0.1% to 1% by weight, relative to the total weight of the composition.

Cationic Polymer (E)

The composition according to the present invention may optionally comprise at least one cationic polymer.

The term “cationic polymer” is intended to mean any polymer comprising cationic groups and/or groups that can be ionized to cationic groups. Preferably, the cationic polymer is hydrophilic or amphiphilic. The preferred cationic polymers are chosen from those that contain units comprising primary, secondary, tertiary and/or quaternary amine groups that may either form part of the main polymer chain or may be borne by a side substituent directly connected thereto. Preferably, the present composition can comprise cationic polysaccharides, in particular cationic celluloses and cationic galactomannan gums. For the purpose of the invention, the present composition preferably comprises cationic celluloses.

Among the cationic celluloses, the present composition comprises cationic cellulose copolymers or cellulose derivatives grafted with a water-soluble quaternary ammonium monomer, which are described, for example, in U.S. Pat. No. 4,131,576, and mention may be made of hydroxyalkyl celluloses, for instance hydroxymethyl-, hydroxyethyl- or hydroxypropylcelluloses grafted, in particular, with a methacryloylethyltrimethylammonium, methacrylamidopropyltrimethylammonium, dimethyldiallylammonium or hydroxypropyltrialkylammonium salt, for example 2-hydroxypropyl trimethyl ammonium chloride. The commercial products corresponding to this definition are more particularly the products sold under the names Celquat® L 200 and Celquat® H 100 by the company National Starch, Polyquaternium-10 (hydroxyethyl cellulose-2-hydroxypropyl trimethyl ammonium chloride ether) such as the product sold under the name Polyquta® 400KC by the company KCI, Polyquaternium-6 (Polydiallyl dimethyl ammonium chloride) such as the product sold under the name Merquat™ 100 polymer from Nalco. Amongst others, Polyquaternium polymers are particularly preferred, e.g., Polyquaternium-7, 11, 15, 16, 22, 39, 47, 55, 67, and 76.

The cationic polymer (E) may be present in the composition according to the present invention in an amount from 0.01 to 5% by weight, preferably from 0.05 to 1% by weight, or preferably from 0.1 to 0.6% by weight, relative to the total weight of the composition.

Solubilizer (F)

The spheric particles of lipophilically modified silica (A) may be incorporated into the composition of the present invention in a water-alcohol system. Accordingly, at least one solubilizer (F) is preferably comprised in the aqueous phase. For the purpose of well dispersion, the solubilizer is preferably a nonionic surfactant or a mixture thereof, more preferably a complex of solubilizers.

Examples of the useful nonionic surfactants may comprise esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives containing oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of C₈-C₂₄ fatty acids, and the oxyalkylenated derivatives thereof; fatty alcohol ethers; the sugar ethers of C₈-C₂₄ fatty alcohols, and mixtures thereof.

Glyceryl esters of fatty acids that may especially be mentioned include glyceryl stearate (glyceryl monostearate, distearate and/or tristearate) (CTFA name: glyceryl stearate), glyceryl oleate, or glyceryl ricinoleate, and mixtures thereof.

Polyethylene glycol esters of fatty acids that may especially be mentioned include polyethylene glycol stearate (polyethylene glycol monostearate, distearate and/or tristearate) and more especially polyethylene glycol 50 OE monostearate (CTFA name: PEG-50 stearate) and polyethylene glycol 100 OE monostearate (CTFA name: PEG-100 stearate), and mixtures thereof.

Mixtures of these surfactants may also be used, for instance the product containing glyceryl stearate and PEG-100 stearate, sold under the name Arlacel® 165 by the company Uniqema, and the product containing glyceryl stearate (glyceryl mono-distearate) and potassium stearate, sold under the name Tegin® by the company Goldschmidt (CTFA name: glyceryl stearate SE).

Fatty acid esters of glucose or of alkylglucose that may be mentioned in particular include glucose palmitate, alkylglucose sesquistearates, for instance methylglucose sesquistearate, alkylglucose palmitates, for instance methylglucose palmitate or ethylglucose palmitate, fatty esters of methylglucoside and more especially the diester of methylglucoside and of oleic acid (CTFA name: methyl glucose dioleate); the mixed ester of methylglucoside and of the oleic acid/hydroxystearic acid mixture (CTFA name: methyl glucose dioleate/hydroxystearate); the ester of methylglucoside and of isostearic acid (CTFA name: methyl glucose isostearate); the ester of methylglucoside and of lauric acid (CTFA name: methyl glucose laurate); the mixture of the monoester and diester of methylglucoside and of isostearic acid (CTFA name: methyl glucose sesquiisostearate); the mixture of the monoester and diester of methylglucoside and of stearic acid (CTFA name: methyl glucose sesquistearate) and in particular the product sold under the name Glucate® SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam® E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate® SSE-20 by the company Amerchol, and the product sold under the name Grillocose® PSE-20 by the company Goldschmidt, and mixtures thereof.

Examples of sucrose esters that may be mentioned include sucrose palmitostearate, sucrose stearate and sucrose monolaurate.

Examples of fatty alcohol ethers that may be mentioned include polyethylene glycol ethers of fatty alcohols containing from 8 to 30 carbon atoms and especially from 10 to 22 carbon atoms, such as polyethylene glycol ethers of cetyl alcohol, of stearyl alcohol or of cetearyl alcohol (mixture of cetyl alcohol and stearyl alcohol). Examples that may be mentioned include ethers comprising from 1 to 200 and preferably from 2 to 100 oxyethylene groups, such as those of CTFA name Ceteareth-20 and Ceteareth-30, and mixtures thereof.

Sugar ethers that may especially be mentioned are alkylpolyglucosides, for example decylglucoside, for instance the product sold under the name Mydol® 10 by the company Kao Chemicals, the product sold under the name Plantaren® 2000 by the company Henkel, and the product sold under the name Oramix® NS 10 by the company SEPPIC; caprylyl/capryl glucoside, for instance the product sold under the name Oramix® CG 110 by the company SEPPIC or under the name Lutensol® GD 70 by the company BASF; laurylglucoside, for instance the products sold under the names Plantaren® 1200 N and Plantacare® 1200 by the company Henkel; cocoglucoside, for instance the product sold under the name Plantacare 818/UP by the company Henkel; cetostearyl glucoside optionally as a mixture with cetostearyl alcohol, sold, for example, under the name Montanov® 68 by the company SEPPIC, under the name Tego-Care® CG90 by the company Goldschmidt and under the name Emulgade® KE3302 by the company Henkel; arachidyl glucoside, for example in the form of the mixture of arachidyl alcohol and behenyl alcohol and arachidyl glucoside, sold under the name Montanov® 202 by the company SEPPIC; cocoylethylglucoside, for example in the form of the mixture (35/65) with cetyl alcohol and stearyl alcohol, sold under the name Montanov® 82 by the company SEPPIC; and mixtures thereof.

Polysorbate 20, PEG-60 hydrogenated castor oil may be additionally mentioned as examples of useful solubilizers.

The solubilizer (F) may be present in the composition according to the present invention in an amount from 0.01% to 5%, such as from 0.05% to 3%, or from 0.1 to 1%, relative to the total weight of the composition including all ranges and subranges therebetween.

Additional Additives

According to various embodiments, the compositions of the present invention are provided for application to keratin materials, such as skin, scalp or hair. In accordance with these embodiments, the compositions of the present invention can comprise various ingredients conventionally useful in compositions for conditioning keratin materials, such as, additional surfactant including nonionic surfactant, anionic surfactant and amphoteric surfactant, active ingredients, humectants, additional fatty substances, antidandruff agents, anti-seborrhoea agents, agents for preventing hair loss and/or for promoting hair regrowth, vitamins and provitamins including panthenol, sunscreens, sequestrants, plasticizers, acidifying agents, opacifiers, pearlescent or nacreous agents, antioxidants, hydroxyacids, fragrances and preserving agents.

A non-exhaustive listing of such ingredients can be found in U.S. patent application publication no. 2004/0170586, the entire contents of which is hereby incorporated by reference. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These additives may be selected variously by the person skilled in the art in order to prepare a composition which has the desired properties, for example, consistency or texture. In particular, the additives, if used, and the amounts thereof are particularly determined according to the specific products/applications thereof, e.g., leave-on conditioner, rinse-off conditioner, skincare conditioner and the like.

These additives may be present in the composition in an amount from 0.01% to 50% relative to the total weight of the composition, including all ranges and subranges therebetween.

Method and Use

The composition according to the present invention can be generally prepared according to the general knowledge of a person skilled in the art. Nevertheless, it is to be understood that a person skilled in the art can choose the method of preparation, on the basis of his/her general knowledge, taking into account the nature of the constituents used, for example, their solubility in the vehicle, and the application envisaged for the compositions or the kit.

According to an embodiment, the composition according to the present invention can be used for caring for keratin materials, especially the scalp, or hair along with scalp. This use may manifest itself as a process for caring for keratin materials, especially the skin, scalp and/or hair, comprising the step applying to said keratin materials the composition of the invention.

The composition according to the present invention is preferably useful to formulate hair conditioner products, scalp care products, or even skincare products. Also, according to the detailed discussions above, it is to be understood that composition, and in turn the conditioner product and/or shampoo product according to the present invention may be free of silicone oil.

The keratin fibers conditioning composition of the present invention can be used in the form of leave-on products, and can be formulated in a wide variety of product forms, including but not limited to creams, lotions, gels, emulsions, mousses and sprays.

The keratin fibers conditioning composition as described above may be used to condition the keratin fibers such as hair along with scalp through conventional methods, depending on the application form of the composition.

For example, when used as a leave-on conditioner after shampooing, the composition of the present invention may be applied through a method comprising following steps:
(i) shampooing hair; and
(ii) applying to the hair an effective amount of the composition for conditioning the hair, without rinsing the hair further.

The invention will be further illustrated by the following examples, which set forth particularly advantageous embodiments.

Notwithstanding that the numerical ranges and parameters setting forth the broad scope of the present invention are approximations, the numerical values set forth in the specific examples are reported as precisely as possible. Any numerical value, however, inherently contain certain errors necessarily resulting from the standard deviation found in their respective measurements. The following examples are intended to illustrate the present invention without limiting the scope as a result.

### EXAMPLES

The ingredient amounts/concentrations in the compositions/formulas described below were expressed in % by weight, relative to the total weight of each composition/formula.

Material:
SILICA SILYLATE 1: AIRLICA® TL-10, available from TOKUYAMA CORPORATION, spheric particles of lipophilically modified silica;
SILICA SILYLATE 2: AEROGEL, available from DOW CHEMICAL, free flowing powder, not spheric particles;

Other materials without specification here were each commercially available.

### Example 1

Composition A, and comparative compositions B, C and D, as Table 1, were prepared:

TABLE 1

| | | Comparative | Comparative | Comparative |
|---|---|---|---|---|
| INCI | Composition A | Composition B | Composition C | Composition D |
| SILICA SILYLATE | 0.5 | | | |
| (AIRLICA TL-10, from | | | | |
| TOKUYAMA CORPORATION) | | | | |
| SILICA SILYLATE | | | | 0.5 |
| (AEROGEL, from DOW | | | | |
| CHEMICAL) | | | | |
| LAURYL | | | 0.5 | |
| METHACRYLATE/ | | | | |
| GLYCOL DIMETHACRYLATE | | | | |
| CROSSPOLYMER | | | | |
| (POLYTRAP 6603 | | | | |
| ADSORBER, from | | | | |
| AMCOL HEALTH & | | | | |
| BEAUTY SOLUTIONS) | | | | |
| CELLULOSE | | 0.5 | | |
| (CELLULOBEADS USF, | | | | |
| from DAITO KASEI | | | | |
| KOGYO) | | | | |
| Ethanol | 20 | 20 | 20 | 20 |
| (ABSOLUTE ETHANOL, | | | | |
| from ANHUI ANTE | | | | |
| FOOD) | | | | |
| MENTHOL | 0.05 | 0.05 | 0.05 | 0.05 |
| (620009 MENTHOL L | | | | |
| PELLETS, from SYMRISE) | | | | |
| PEG-60 | 0.3 | 0.3 | 0.3 | 0.3 |
| HYDROGENATED CASTOR OIL | | | | |
| (EUMULGIN CO 60, from | | | | |
| BASF) | | | | |
| POLYQUATERNIUM-11 | 0.2 | 0.2 | 0.2 | 0.2 |
| (GAFQUAT 755, from ISP) | | | | |
| VP/VA COPOLYMER | 0.4 | 0.4 | 0.4 | 0.4 |
| (LUVISKOL VA 64 | | | | |
| POUDRE, from BASF) | | | | |
| CARBOMER | 0.1 | 0.1 | 0.1 | 0.1 |
| (CARBOPOL ULTREZ 10 | | | | |
| POLYMER, from LUBRIZOL) | | | | |
| WATER | QS to 100 | QS to 100 | QS to 100 | QS to 100 |

The above listed compositions A-D were prepared according to known manufacturing method of field.

### Example 2

The effect of the composition on the inhibition of grease was determined with sebum migration test, comprising the steps of:

Step 1: cleaning hair swatches with standard shampoo DOP and washed with test products according to known procedure RC-MCI-OIP-003B

Step 2: applying 100 μL colored artificial sebum (red) on selected 3 cm area on top region of hair swatch.

Step 3: hanging the hair swatch on iron stand in 30° C., 50% humidity condition.

Step 4: taking images of hair swatch at 1 h, 2 h, 4 h, 6 h, and 24 h.

Step 5: using image analysis to calculate the total sebum migration area at each time point.

The sebum migration results were provided as follows:

TABLE 2

| | | Comparative | Comparative | Comparative |
|---|---|---|---|---|
| Properties | Composition A | Composition B | Composition C | Composition D |
| Oil absorption | 238.53 mm² | 281.37 mm² | 256.93 mm² | 256.55 mm² |
| (Sebum migration | | | | |
| area) | | | | |

For the sebum migration test, less sebum migration area means more inhibition to the grease. Thus, it was clear to see comparing with other particles, e.g., those not in spheric shape, or not lipophilically modified silica, the spheric particles of lipophilically modified silica homogeneously when being dispersed in the medium/solvent, can bring unexpectedly enhanced grease inhibition.

### Example 3

6 volunteers between age of 25 and 45 applied the compositions A and B on the hair and scalp every day for 3 days. Scores of sticky or greasy feeling before the application of the compositions A and B were given as baselines, the scores of sticky or greasy feeling after application of the compositions A and B were given every day, the mean difference was shown in Table 4.

Volunteers gave scores to the greasy feeling, where:
0 meant unchanged;
1 meant fairly improved;
2 meant significantly improved; and
3 meant substantially free of greasy feeling.

The average scores of the 6 volunteers were counted instant status, 24 h, and 48 h, and were calculated for the averages for every day. The average scores for the total 3 days were provided in Table 3 below.

TABLE 3

| | | Day 2 | Day 3 |
|---|---|---|---|
| | Consumer 1-6 | Consumer 1-6 | Consumer 1-6 |
| | Instant average | 24 h average | 48 h average |
| Composition A | 2.0 | 2.0 | 2.0 |
| Comparative | 1.0 | 1.0 | 1.0 |
| composition B | | | |

Note:
for convenience, the each average score in Table 4 was rounded to an integer.

Referring to Table 3, less the score, less the sticky or greasy feeling was. It was observed that the composition A showed a very good and long term anti-sticky or greasy feeling.

1: A composition for conditioning keratin materials, comprising:
(A) spheric particles of lipophilically modified silica homogeneously dispersed in a medium/solvent,
(B) at least one medium/solvent selected from water and alcohols, and
(C) at least one conditioning active agent.

2: The composition according to claim 1, wherein the spheric particles of lipophilically modified silica (A) comprises silica silylate and silica dimethyl silylate.

3: The composition according to claim 1, wherein the spheric particles of lipophilically modified silica (A) is present in an amount ranging from 0.01% to 10% by weight relative to the total weight of the composition.

4: The composition according to claim 1, wherein the alcohol is selected from the group consisting of C2-C10 monoalcohol, C2-C10 diols, and C3-C6 polyols containing more than two hydroxyl functions.

5: The composition according to claim 1, wherein the medium/solvent (B) comprises both water and alcohol, in which water is present in the composition in an amount from 40% to 99% by weight relative to the weight of the composition, and/or alcohol is present in the composition in an amount from 0.1% to 40% by weight, relative to the weight of the composition.

6: The composition according to claim 1, wherein the conditioning active agent (C) is selected from the group consisting of non-silicone cationic surfactants, fatty esters, and menthol and derivatives thereof.

7: The composition according to claim 1, wherein the conditioning active agent (C) is present in an amount ranging from 0.001% to 1% by weight relative to the total weight of the composition.

8: The composition according to claim 1, further comprising at least one thickening agent (D), selected from the group consisting of carboxylic acid polymers, crosslinked polyacrylate polymers, polyacrylamide polymers, polysaccharides, and gums, or a mixture thereof.

9: The composition according to claim 8, wherein the at least one thickening agent (D) is present in the composition in an amount from 0.01% to 5% by weight relative to the total weight of the composition including all ranges and subranges therebetween.

10: The composition according to claim 1, further comprising at least one cationic polymer (E), selected from the group consisting of cationic cellulose copolymers and cellulose derivatives grafted with a water-soluble quaternary ammonium monomer.

11: The composition according to claim 10, wherein the cationic polymer (E) is present in the composition in an amount from 0.01 to 5% by weight relative to the weight of the composition, including all ranges and subranges therebetween.

12: The composition according to claim 1, further comprising at least one solubilizer (F), selected from the group consisting of glyceryl esters of C18-C24 fatty acids, oxyalkylenated derivatives thereof; polyethylene glycol esters of C8-C24 fatty acids, oxyalkylenated derivatives thereof; sorbitol esters of C8-C24 fatty acids, oxyalkylenated derivatives thereof; sorbitan esters of C8-C24 fatty acids, oxyalkylenated derivatives thereof; sugar esters of C8-C24 fatty acids, oxyalkylenated derivatives thereof; fatty alcohol ethers; the sugar ethers of C8-C24 fatty alcohols, and mixtures thereof.

13: The composition according to claim 12, wherein the solubilizer (F) is present in the composition in an amount from 0.01% to 5%, relative to the weight of the composition, including all ranges and subranges therebetween.

14: The composition according to claim 1, which does not comprise a silicone-based oil.

15: A leave-on keratin material care product comprising the composition according to claim 1, suitable for caring for skin, scalp and/or hair.
